(19) United States
(12) Patent Application Publication
(10) Pub. No.: US 2011/0306501 A1
(43) Pub. Date: Dec. 15, 2011

(54) ACTIVE INGREDIENT-COMPRISING MICROCAPSULES WITH A METAL OXIDE-CONTAINING SHELL

(75) Inventors: Jing Dreher, Stuttgart (DE); Matthias Laubender, Schifferstadt (DE); Michael Kutschera, Neustadt (DE)

(73) Assignee: BASF SE, Ludwigshafen (DE)

(21) Appl. No.: 13/155,735

(22) Filed: Jun. 8, 2011

Related U.S. Application Data
(60) Provisional application No. 61/352,844, filed on Jun. 9, 2010.

(51) Int. Cl. A01N 25/28; A61K 8/02; C11D 17/00; B01J 13/02; C09K 15/32; A23K 1/00; A23L 1/29; A61K 9/50; C09K 21/06

(52) U.S. Cl. 504/359; 424/490; 424/401; 510/418; 510/508; 512/4; 252/609; 252/400.1; 252/400.52; 426/89; 264/4.1

(57) ABSTRACT

The present invention relates to microcapsules with a core/shell structure which comprise at least one low molecular weight, anionic surfactant and have a metal oxide-containing shell, to a process for producing such microcapsules with a core/shell structure, to the use of the microcapsules with the core/shell structure and to preparations comprising the microcapsules with the core/shell structure.

The present invention relates to microcapsules with a core/shell structure which comprise at least one low molecular weight, anionic surfactant and have a metal oxide-containing shell, to a process for producing such microcapsules with a core/shell structure, to the use of the microcapsules with the core/shell structure and to preparations comprising the microcapsules with the core/shell structure.

The encapsulation of active ingredients is carried out for various reasons. For example, encapsulation can increase the storage stability of those active ingredients which are sensitive to light, oxygen or moisture. In the case of pharmaceutical active ingredients, the release of active ingredient can be influenced in a targeted manner by the encapsulation. Or liquid substances can be handled following encapsulation in the form of a pourable powder. In the field of detergents and cleaners or textile care compositions, the encapsulation of flavorings, aroma substances or fragrances serves on the one hand to prevent a reaction of these encapsulated substances with atmospheric oxygen or other chemicals present in the formulations, such as, for example, bleaches. A further problem is the sometimes high volatility of the scents or fragrances, which leads to a large part of the scent or fragrance amount originally admixed with the detergent or cleaner having already volatilized prior to the point of use. To overcome the discussed problems, it has already been proposed to incorporate the scents or fragrances into the detergents or cleaners in microencapsulated form. Microcapsules of this type have already been described.

WO 2005/009604 A1 describes microcapsules with a high active ingredient content in which a core which comprises an active ingredient is surrounded by a shell, where the shell comprises an inorganic polymer.

WO 2007/015243 describes a process for producing microcapsules in which solid hydrophobic active ingredients are surrounded by several shells of silica gel.

DE 10 2008 030 662 describes coated scented and/or aroma particles which in each case comprise a particle core comprising silicates and fragrances and/or aromas and a sheath comprising water-soluble silicates.

WO 2009/090169 describes microcapsules, the core of which comprises a scent or fragrance, and the shell of which has been constructed by polymerization of acrylic and/or methacrylic acid esters and at least two different bi- or polyfunctional monomers.

Despite the prior art described at the start, there is furthermore a need for capsules which exhibit improved stability against unintentional rupture of the shell and have a denser, less porous shell in order to prevent the active ingredient from escaping or volatilizing. On the other hand, the shell must not be so stable that release of the scents or fragrances upon normal mechanical stress barely takes place, or does not take place at all. Furthermore, the capsules should not comprise undesired by-products, such as, for example, remains of unreacted monomers. Finally, the process for producing the capsules should be as broadly useable as possible and simple to carry out. The process for producing the capsules should be stable both to thermal and also to mechanical stresses.

It was therefore the edition of the present invention to provide active ingredient-containing capsules with improved stability with regard to the protection of the active ingredient against undesired volatilization and to be able to produce the novel active ingredient-containing capsules by a simple and robust process.

This object is achieved by microcapsules with a core/shell structure which comprise at least one low molecular weight, anionic surfactant, wherein each microcapsule comprises in the inside a core which comprises at least one sparingly water-soluble or water-insoluble organic active ingredient, and, directly around the core, has a shell, where the shell comprises at least one metal oxide and at least one water-soluble polymer.

The mass fraction of the core relative to the total mass of the microcapsules according to the invention is usually greater than 10% by weight, preferably greater than 20% by weight, particularly preferably in the range from 25 to 80% by weight, very particularly preferably in the range from 30 to 60% by weight. The percentages refer to a statistical average value determined over a large number of microcapsules.

The microcapsules according to the invention with core/shell structure comprise in the inside in each case a core which comprises at least one sparingly water-soluble or water-insoluble organic active ingredient. The core may either be liquid or solid at 20° C. If the core is a solid at 20° C., this solid may be crystalline, partially crystalline or amorphous. If the core is a liquid at 20° C., this liquid may be a homogeneous phase or a suspension. Preferably, the core of the microcapsules according to the invention is a liquid at 20° C.

The core in the inside of the microcapsules according to the invention often comprises, besides the at least one sparingly water-soluble or water-insoluble organic active ingredient, at least one sparingly water-soluble or water-insoluble organic auxiliary which serves, for example, to adjust the concentration of the active ingredient, to alter the release profile of the active ingredient or simply to dissolve or suspend the active ingredient. Consequently, the concentration of the active ingredient in the core of the microcapsules according to the invention can be varied over a wide range according to the field of use and the properties of the active ingredient, such as, for example, its solubility in the organic auxiliary.

On account of its composition, the core preferably exhibits hydrophobic properties, i.e. the core is only sparingly water-soluble or is water-insoluble.

The microcapsules according to the invention usually have an average particle size (d50 value) of less than 100 μm, preferably less than 10 μm, particularly preferably an average particle size of from 0.05 μm to 5 μm, in particular from 0.1 μm to 2 μm.

The d50 value is defined in that 50% by weight of the particles have a diameter which is less than the value which corresponds to the d50 value, and 50% by weight of the particles have a diameter which is larger than the value which corresponds to the d50 value. The d50 value can be read off from a particle size distribution curve, as can be generated, for example, by means of light scattering in accordance with ISO 13320-1 (e.g. Microtrac S3500 Bluewave from Microtrac).

The microcapsules according to the invention particularly preferably have an average particle size of less than 2 μm.

The average particle size of the microcapsules and the thickness of the shells can be determined by means of TEM (transmission electron microscopy). The average particle size can be determined using the methods of light scattering (static and dynamic light scattering).

The shape of the cores in the microcapsules according to the invention is arbitrary and can be, for example, irregular or spherical, preferably spherical.

The microcapsules with core/shell structure according to the invention comprise at least one low molecular weight, anionic surfactant, where the molar mass of the low molecular weight, anionic surfactants is usually less than 1000 g/mol, preferably less than 500 g/mol. Besides a nonpolar, fat-soluble part, anionic surfactants have a polar, negatively charged part, such as, for example, a carboxylate radical, a sulfonate radical, a sulfate radical or a phosphate radical. Typical representatives of low molecular weight, anionic surfactants are, for example, alkyl carboxylates, alkylbenzenesulfonates, secondary alkanesulfonates, alkylphenol sulfate or fatty alcohol sulfates. Typical representatives of the various classes of anionic surfactants are listed in WO 2009/090169, page 10, line 37 to page 12, line 6. Particular preference is given to microcapsules in which the low molecular weight, anionic surfactant is a fatty alcohol sulfate or an alkylphenol ether sulfate. The salts of the preferred fatty alcohol sulfates have the following formula: CₙH₂ₙ₊₁OSO₃⁻M⁺ where n is 8 to 18, in particular 10 to 16 and M is Na or K, in particular Na.

Particularly preferably, the microcapsules with core/shell structure according to the invention comprise, besides the at least one low molecular weight, anionic surfactant, also at least one nonionic surfactant. Nonionic surfactants are interface-active substances with an uncharged, polar, hydrophilic, water-solubilizing head group that carries no ion charge in the neutral pH range (in contrast to anionic and cationic surfactants), which adsorbs at interfaces and aggregates to give neutral micelles above the critical micelle concentration (cmc). Depending on the type of hydrophilic head group, a distinction can be made between (oligo)oxyalkylene groups, in particular (oligo)oxyethylene groups (polyethylene glycol groups), to which the fatty alcohol polyglycol ethers (fatty alcohol alkoxylates), alkylphenol polyglycol ethers and fatty acid ethoxylates, alkoxylated triglycerides and mixed ethers (polyethylene glycol ethers alkylated on both sides) belong; and carbohydrate groups, to which e.g. the alkyl polyglucosides and fatty acid N-methylglucamides belong. Typical representatives of the different classes of nonionic surfactants are listed in WO 2009/090169, page 13, line 17 to page 15, line 9.

Particularly preferred nonionic surfactants are sorbitan fatty acid esters with polyethylene glycol radicals, so-called polysorbates (Tween® grades), such as, for example, polyoxyethylene(20) sorbitan monolaurate.

Preferably, the weight ratio between the anionic surfactant, in particular a fatty alcohol sulfate of the formula CₙH₂ₙ₊₁OSO₃⁻M⁺, and the nonionic surfactant, is 1:1 to 5:1, particularly preferably 1:1 to 3:1.

During the production of the microcapsules according to the invention, the weight fraction of the surfactants used, based on the mass of the oil phase, is preferably in the range from 0.5% to 20%, in particular in the range from 1% to 5%.

Suitable sparingly water-soluble or water-insoluble organic active ingredients are those organic compounds which are used, for example, for the food and animal nutrition sector, for pharmaceutical and cosmetic applications, in the field of crop protection, in detergents and cleaners, textile care compositions or in the field of plastics additives. The sparingly water-soluble or water-insoluble organic active ingredient may, however, also be an explosive, a wax or an insect repellant. The microcapsules according to the invention can advantageously be used in all of the applications in which the active ingredient is to be temporarily or permanently separated from the surrounding area.

The organic active ingredients are chemical compounds which usually comprise both carbon and also hydrogen.

A sparingly water-soluble organic active ingredient is usually a chemical compound, the solubility of which in water at 20° C. is less than 10 g/l, preferably less than 1 g/l, particularly preferably less than 0.1 g/l.

Active ingredients which are used in the food and animal nutrition sector are, inter alia, lipophilic vitamins, such as, for example, tocopherol, vitamin A, and derivatives thereof, vitamin D and derivatives thereof, vitamin K and derivatives thereof, vitamin F and derivatives thereof, or saturated and unsaturated fatty acids, and also derivatives and compounds thereof, natural and synthetic flavorings, aroma substances and fragrances and lipophilic dyes, such as, for example, retinoids, flavonoids or carotenoids.

Active ingredients which are used in the pharmaceutical sector are, inter alia, anesthetics and narcotics, anticholinergics, antidepressants, psychostimulants and neuroleptics, antiepileptics, antimycotics, antiphlogistics, bronchodilators, cardiovascular medicaments, cytostatics, hyperemics, antilipemics, spasmolytics, testosterone derivatives, tranquilizers or virustatics.

Active ingredients which are used in the field of cosmetics are, for example, perfume oils, organic UV filters, dyes, organic pigments or care substances such as vitamin E acetate.

Preferred dyes which can be used as active ingredients in the microcapsules according to the invention are natural or synthetic dies which are approved in the field of nutrition or cosmetics, as are described, for example, in WO 2005/009604 A1 on page 9, lines 18 to 30.

Active ingredients for the crop protection sector are lipophilic agrochemicals, such as, for example, insecticides, fungicides, pesticides, nematicides, rodenticides, molluscicides, growth regulators and herbicides.

The term pesticide (or agrochemical active ingredient) refers to at least one active ingredient selected from the group of fungicides, insecticides, nematicides, herbicides, rodenticides, safeners and/or growth regulators. Preferred pesticides are fungicides, insecticides, rodenticides and herbicides. Mixtures of pesticides of two or more of the aforementioned classes can also be used. The person skilled in the art is familiar with such pesticides, which can be found, for example, in Pesticide Manual, 14th ed. (2006), The British Crop Protection Council, London.

Active ingredients which are used in the field of detergents and cleaners or of textile care compositions are, for example, aroma substances or fragrances, disinfectants, dies, bleaches, biocides, organic solvents or perfumes.

Active ingredients which are used in the field of plastics additives are, for example, photostabilizers, such as UV stabilizers, flame retardants or antioxidants.

Preferably, the sparingly water-soluble or water-insoluble organic active ingredient which is present in the core of the microcapsules according to the invention is an aroma chemical, in particular a flavoring, scent or fragrance.

An aroma chemical, in particular a scent or fragrance, is understood as meaning all organic substances which have a desired olfactory property and are essentially nontoxic. These include inter alia all scents or fragrances customarily used in detergent or cleaner compositions or in perfumery. They may be compounds of natural, semisynthetic or synthetic origin. Preferred scents or fragrances can be assigned to the substance classes of the hydrocarbons, aldehydes or esters. The scents or fragrances also include natural extracts and/or essences which can comprise complex mixtures of constituents, such as orange oil, lemon oil, rose extract, lavender, musk, patchouli, balsam essence, sandalwood oil, pine oil and cedar oil.

Nonlimiting examples of synthetic and semisynthetic scents or fragrances are: 7-acetyl-1,2,3,4,5,6,7,8-octahydro-1,1,6,7-tetramethylnaphthalene, α-ionone, β-ionone, γ-ionone, α-isomethylionone, methyl cedrylone, methyl dihydrojasmonate, methyl 1,6,10-trimethyl-2,5,9-cyclododecatrien-1-yl ketone, 7-acetyl-1,1,3,4,4,6-hexamethyltetralin, 4-acetyl-6-tert-butyl-1,1-dimethylindane, hydroxyphenylbutanone, benzophenone, methyl β-naphthyl ketone, 6-acetyl-1,1,2,3,3,5-hexamethylindane, 5-acetyl-3-isopropyl-1,1,2,6-tetramethylindane, 1-dodecanal, 4-(4-hydroxy-4-methylpentyl)-3-cyclohexene-1-carboxaldehyde, 7-hydroxy-3,7-dimethyloctanal, 10-undecen-1-al, isohexenylcyclohexylcarboxaldehyde, formyltricyclodecane, condensation products of hydroxycitronellal and methyl anthranilate, condensation products of hydroxycitronellal and indole, condensation products of phenylacetaldehyde and indole, 2-methyl-3-(para-tert-butylphenyl)propionaldehyde, ethylvanillin, heliotropin, hexylcinnamaldehyde, amylcinnamaldehyde, 2-methyl-2(isopropylphenyl)propionaldehyde, coumarin, γ-decalactone, cyclopentadecanolide, 16-hydroxy-9-hexadecenolactone, 1,3,4,6,7,8-hexahydro-4,6,6,7,8,8-hexamethylcyclopenta-γ-2-benzopyran, β-naphthol methyl ether, ambroxan, dodecahydro-3a,6,6,9a,tetramethylnaphtho[2,lb]furan, cedrol, 5-(2,2,3-trimethylcyclopent-3-enyl)-3-methylpentan-2-ol, 2-ethyl-4-(2,2,3-trimethyl-3cyclopenten-1-yl)-2-buten-1-ol, caryophyllene alcohol, tricyclodecenyl propionate, tricyclodecenyl acetate, benzyl salicylate, cedryl acetate and tert-butyl cyclohexyl acetate.

Further examples of scents or fragrances that can be used in the microcapsules according to the invention are described, for example, in U.S. Pat. Nos. 6,143,707, 5,089,162, EP 1 360 270, DE 10 2008 030 662 paragraph 0025 to 0036 and WO 2009/027957. A detailed description of the various aroma chemicals used most often which can likewise be used in the microcapsules according to the invention as sparingly water-soluble or water-insoluble organic active ingredient can be found in the book “Common Fragrance and Flavor Materials” by H. Surburg and J. Panten, 5th completely revised and expanded edition—February 2006, Wiley-VCH, Weinheim.

Particular preference is given to: hexylcinnamaldehyde, 2-methyl-3-(tert-butylphenyl)propionaldehyde, 7-acetyl-1,2,3,4,5,6,7,8-octahydro-1,1,6,7-tetramethyl-naphthalene, benzyl salicylate, 7-acetyl-1,1,3,4,4,6-hexamethyltetralin, para-tert-butyl cyclohexylacetate, methyl dihydrojasmonate, β-naphthol methyl ether, methyl β-naphthyl ketone, 2-methyl-2-(para-isopropylphenyl)propionaldehyde, 1,3,4,6,7,8-hexahydro-4,6,6,7,8,8-hexamethylcyclpenta-γ-2-benzopyran, dodecahydro-3a,6,6,9a-tetramethylnaphtho[2,lb]furan, anisaldehyde, coumarin, cedrol, vanillin, cyclopentadecanolide, tricyclodecenyl acetate and tricyclodecenyl propionates.

Other scents are essential oils, resinoids and resins from a large number of sources, such as Peru balsam, olibanum resinoid, styrax, labdanum resin, mace, cassia oil, benzoin resin, coriander and lavandin. Further suitable fragrances are: phenylethyl alcohol, terpineol, linalool, linalyl acetate, geraniol, nerol, 2-(1,1-dimethylethyl)cyclohexanol acetate, benzyl acetate and eugenol.

The scents or fragrances can be used as pure substances or in a mixture with one another.

In principle, the sparingly water-soluble or water-insoluble organic active ingredient, preferably the aroma chemical, in particular the scent or fragrance, can be a liquid or a solid at 20° C., where the solid may even also be present in a suitable lipophilic solvent, such as an oil, in dissolved form or as suspension. Thus, e.g. when using scents or fragrances that are solid at room temperature, the use of a hydrophobic material that is liquid at room temperature as solution or dispersant is advantageous.

Preferably, the sparingly water-soluble or water-insoluble organic active ingredient used in the microcapsule according to the invention is a liquid at 20° C.

To increase the hydrophobicity of a scent or fragrance, a further hydrophobic material can be added to this scent or fragrance.

Preferably, the sparingly water-soluble or water-insoluble organic active ingredient, in particular the scent or fragrance or the mixture of scents or fragrances, constitutes 1 to 100% by mass, preferably 20 to 100% by mass, of the hydrophobic core material. The hydrophobic material is liquid at temperatures below 100° C., preferably at temperatures below 60° C. and particularly preferably at room temperature.

Besides the sparingly water-soluble and water-insoluble active ingredient, the core of the microcapsules according to the invention can also comprise hydrophobic auxiliaries such as oils or solvents which are customarily used in the respective fields of application.

The hydrophobic auxiliaries which can be used as constituent of the core of the microcapsule according to the invention include all types of oils, such as vegetable oils, animal oils, mineral oils, paraffins, chloroparaffins, fluorinated hydrocarbons and other synthetic oils.

Typical and nonexhaustive examples are sunflower oil, rapeseed oil, olive oil, peanut oil, soyabean oil, kerosene, benzene, toluene, butane, pentane, hexane, cyclohexane, chloroform, tetrachloromethane, chlorinated diphenyls and silicone oil. It is also possible to use hydrophobic materials with a high boiling point, e.g. diethyl phthalate, dibutyl phthalate, diisohexyl phthalate, dioctyl phthalate, alkylnaphthalene, dodecylbenzene, terphenyl, partially hydrogenated terphenyls, ethylhexyl palmitate, capric/caprylic triglyceride, PPG-2 myristyl ether propionate, PPG-5 ceteth-20; C₁₂₋₁₅-alkyl benzoate, mineral oil (CAS: 8042-47-5), cetearyl ethylhexanoate, dimethicone, polyisobutylene (e.g. BASF: Glisopal®, Oppanol®). Customary oil components in cosmetics are, for example, paraffin oil, glyceryl stearate, isopropyl myristate, diisopropyl adipate, cetylstearyl 2-ethylhexanoate, hydrogenated polyisobutene, vaseline, caprylic/capric triglycerides, microcrystalline wax, lanolin and stearic acid. However, this list is exemplary and nonexhaustive.

The microcapsules according to the invention have a shell directly around the core. This shell comprises at least one metal oxide and at least one water-soluble polymer.

The metal oxide of the shell is usually a metal oxide insoluble in water (pH 7). The metal oxide of the shell is preferably a metal oxide which is derived from a metal from which water-soluble, especially readily water-soluble salts of this metal in oxidation state +II, +III or +IV are available. In these salts, the metal is particularly preferably present as a doubly or triply charged cation.

Within the context of the present invention, readily soluble salts in water at 20° C. have a solubility of at least 10 g/l, preferably of at least 100 g/l, particularly preferably of at least 200 g/l.

According to the invention, the metal oxide of the shell is preferably an oxide of the metals zinc, cerium, zirconium, aluminum, magnesium, iron, manganese, nickel or cobalt. The metal oxide of the shell is particularly preferably zinc oxide.

Besides the metal oxide, the shell of the microcapsules according to the invention comprises at least one water-soluble polymer. Examples of such water-soluble polymers are polyacrylates, polyaspartic acid, polyethers, polyvinylpyrrolidones, and corresponding copolymers thereof.

Preferably, the water-soluble polymer in the side chains comprises carboxy or carboxylate groups, such as, for example in the case of polyacrylates or polyaspartic acid. Particular preference is given to polyacrylates.

Polyacrylates particularly preferred according to the invention are polymers based on at least one α,β-unsaturated carboxylic acid, for example acrylic acid, methacrylic acid, dimethacrylic acid, ethacrylic acid, maleic acid, citraconic acid, methylenemalonic acid, crotonic acid, isocrotonic acid, fumaric acid, mesaconic acid and itaconic acid. Preferably, polyacrylates based on acrylic acid, methacrylic acid, maleic acid or mixtures thereof are used.

Besides the at least one α,β-unsaturated carboxylic acid, the polyacrylates can also comprise further comonomers which are polymerized into the polymer chain, for example the esters, amides and nitriles of the carboxylic acids stated above, e.g. methyl acrylate, ethyl acrylate, methyl methacrylate, ethyl methacrylate, hydroethyl acrylate, hydroxypropyl acrylate, hydroxybutyl acrylate, hydroxyethyl methacrylate, hydroxypropyl methacrylate, hydroxyisobutyl acrylate, hydroxyisobutyl methacrylate, monomethyl maleate, dimethyl maleate, monoethyl maleate, diethyl maleate, 2-ethylhexyl acrylate, 2-ethylhexyl methacrylate, acrylamide, methacrylamide, N-dimethylacrylamide, N-tert-butylacrylamide, acrylonitrile, methacrylonitrile, dimethylaminoethyl acrylate, diethylaminoethyl acrylate, diethylaminoethyl methacrylate, and the salts of the last-mentioned basic monomers with carboxylic acids or mineral acids, and also the quaternized products of the basic (meth)acrylates.

Many of the polyacrylates to be used according to the invention are commercially available under the trade name Sokalan® (BASF SE). Very particular preference is given to a polyacrylate which is obtained from pure acrylic acid.

The molecular weight of the polyacrylates particularly preferred according to the invention is generally in the range from 800 to 250 000 g/mol, preferably in the range from 1000 to 100 000 g/mol, particularly preferably in the range from 1000 to 20 000 g/mol.

The present invention also provides a process for producing microcapsules with a core/shell structure which comprise at least one low molecular weight, anionic surfactant, wherein each microcapsule comprises in the inside a core which comprises at least one sparingly water-soluble or water-insoluble organic active ingredient, and, directly around the core, has a shell, where the shell comprises at least one metal oxide and at least one water-soluble polymer, comprising the process steps

i) preparing an oil-in-water emulsion by emulsifying an oil phase which comprises at least one sparingly water-soluble or water-insoluble organic active ingredient in a water phase which comprises at least one low molecular weight, anionic surfactant and at least one water-soluble salt of a metal in oxidation state +II, +III or +IV, using shear forces,

ii) washing the oil-in-water emulsion to remove salt dissolved free in the water phase and anionic surfactant until the aqueous phase has a conductivity of less than 2.0 mS/cm,

iii) producing solid metal oxide at the oil phase boundary from the water-soluble salt of the metal in oxidation state +II, +III or +IV, which is bonded in the region of the oil/water phase boundary by adding an aqueous solution which comprises a strong base and a water-soluble polymer to the emulsion washed in process step ii), and

iv) if appropriate, purifying and/or isolating the microcapsules with core/shell structure produced in process step iii).

Preferred embodiments with regard to the sparingly water-soluble or water-insoluble active ingredient, with regard to the low molecular weight anionic surfactant, with regard to the metal oxide, with regard to the water-soluble polymer, and also preferred embodiments with regard to dimensions and mass fractions of the various constituents of the microcapsules with a core/shell structure can be found in the explanations already given at the start.

In process step i), the preparation of an oil-in-water emulsion by emulsifying an oil phase which comprises at least one sparingly water-soluble or water-insoluble organic active ingredient in a water phase which comprises at least one low molecular weight, anionic surfactant and at least one water-soluble salt of a metal in oxidation state +II, +III or +IV using shear forces is described.

The methods of producing emulsions using shear forces are known in principle to the person skilled in the art. Thus, for example, fanta bowl and pestle, high-speed stirrers, high-pressure homogenizers, shakers, vibratory mixers, ultrasound generators, emulsifying centrifuges, colloid mills or atomizers can be used for generating emulsions. The person skilled in the art in each case selects the suitable method and the appropriate emulsifying tool depending on the desired result, for example the desired droplet size in the emulsion, and depending on the physicochemical properties of the selected feed materials, for example their viscosity and also their thermal stability.

In process step i), the oil phase fraction in the emulsion is preferably from 1 to 50% by weight, particularly preferably from 10 to 40% by weight, based on the total mass of the emulsion.

The weight fraction of the surfactants used in process step i), based on the mass of the oil phase, is preferably in the range from 0.5% to 20%, in particular in the range from 1% to 5%.

The water-soluble salt of a metal in oxidation state +II, +III or +IV, in particular oxidation state +II or +III, used in process step i) is preferably a salt of the specified oxidation states that is readily soluble in water. Examples of such preferred salts are, for example, the halides, acetates or nitrates of the metals zinc, cerium, tin, aluminum, magnesium, iron, manganese, nickel or cobalt, in particular of zinc. In process step i), particular preference is given to using zinc chloride, zinc nitrate or zinc acetate as water-soluble salt.

In process step i), the concentration of the used water-soluble salt of a metal in oxidation state +II, +III or +IV in the water phase is generally in the range from 0.05 to 1 mol/l, preferably in the range from 0.1 to 0.5 mol/l, particularly preferably in the range from 0.2 to 0.4 mol/l.

In process step i), the molar ratio between the low molecular weight anionic surfactant and the water-soluble salt of a metal in oxidation state +II, +III or +IV is preferably in the range from 1:10 to 1:1, preferably in the range from 1:8 to 1:3.

After preparing the emulsion, it can also be stirred for some time. One can imagine that the formation of “multilayered metal ion sheaths” of the metal in oxidation state +II, +III or +IV around the oil droplets surrounded externally with the anionic surfactant can in some circumstances require some time. Preferably, the afterstirring time is between 1 and 10 hours.

In process step ii), in order to remove salt dissolved free in the water phase and anionic surfactant, the oil-in-water emulsion produced in process step i) is washed until the aqueous phase has a conductivity of less than 2.0 mS/cm.

Process step ii) can be realized by centrifugation, filtration, evaporation of the solvent, resuspension and dialysis methods. Particularly preferably, a cross-flow filtration is carried out as filtration method.

The emulsion is washed until the aqueous phase has a conductivity of less than 2.0 mS/cm, preferably a conductivity in the range from 0.1 to 0.5 mS/cm. The measurement of the conductivity is known to the person skilled in the art and can be undertaken directly in the emulsion to be purified using a standard commercial conductivity measuring device.

In process step iii), by adding an aqueous solution which comprises a strong base and a water-soluble polymer to the emulsion washed in process step ii), solid metal oxide is generated at the oil phase boundary from the water-soluble salt of the metal in oxidation state +II, +III or +IV, which is bonded in the region of the oil/water phase boundary.

The strong bases to be used according to the invention are generally any desired substances which are able, in aqueous solution depending on their concentration, to generate a pH of from about 8 to about 13, preferably from about 9 to about 12.5. These may be, for example, metal oxides or hydroxides and also ammonia or amines. Preference is given to using alkali metal hydroxides such as sodium hydroxide or potassium hydroxide, alkaline earth metal hydroxides such as calcium hydroxide or ammonia. Particular preference is given to using sodium hydroxide, potassium hydroxide or ammonia, in particular sodium hydroxide, as strong base.

The concentration of the strong base in the aqueous solution which is added in process step iii) to the emulsion washed in process step ii) is generally selected such that preferably a hydroxide ion concentration in the range from 0.1 to 2 mol/l, particularly preferably from 0.2 to 1 mol/l and in particular from 0.4 to 0.8 mol/l, is established in the aqueous solution.

Preferably, the quantitative amount of hydroxide ions in the aqueous solution is selected such that, at least theoretically, the metal ions of the metal in oxidation state +II, +III or +IV present in the emulsion could be completely present in the form of the corresponding hydroxides M(OH)₂, M(OH)₃ or M(OH)₄ when the addition of the aqueous solution is complete.

The concentration of the water-soluble polymer, in particular of the polyacrylates, in the aqueous solution which is added in process step iii) to the emulsion washed in process step ii) is generally in the range from 0.1 to 20 g/l, preferably in the range from 1 to 10 g/l, particularly preferably in the range from 1.5 to 5 g/l. The water-soluble polymers to be used according to the invention must naturally have a corresponding solubility in water.

One preferred embodiment of the process according to the invention is characterized in that the precipitation of the metal oxide, metal hydroxide and/or of the metal oxide hydroxide and generation of the solid metal oxide takes place in the presence of a polyacrylate which is obtained from pure acrylic acid. In one particularly preferred embodiment of the invention, Sokalan® PA 15 (BASF SE), the sodium salt of a polyacrylic acid (Mₙ: ca. 1200 g/mol; pH: ca. 8; K value in accordance with ISO 1628-1, 1% in distilled water: ca. 15) is used.

The addition of the aqueous solution which comprises the strong base and the water-soluble polymer to the emulsion washed in process step ii) is usually carried out at a temperature in the range from 0 to 100° C., preferably 10 to 60° C., in particular 30 to 45° C.

The addition of the aqueous solution which comprises the strong base and the water-soluble polymer to the washed emulsion can in principle take place as quickly as desired. Preferably, the addition is undertaken in a uniformly distributed manner over more than 30 minutes.

Particularly preferably, in process step iii), the addition of the aqueous solution which comprises a strong base and a water-soluble polymer is undertaken in a uniformly distributed manner over a period from 2 to 5 hours.

According to the current level of knowledge, the water-soluble polymer serves to increase the rate of dehydration of the primarily formed metal hydroxide to the metal oxide. In the present process, it is possible to dispense with the calcination processes often used in the conversion of metal hydroxides to the corresponding metal oxides at temperatures above 100° C.

The suspension of microcapsules formed in process step iii) can additionally be stabilized by adding further additives, such as nonionic, anionic or cationic polymers or surfactants.

The microcapsules with core/shell structure produced in process step iii) can be further purified and/or isolated in a subsequent process step iv). Corresponding purification and isolation methods are known to the person skilled in the art, such as, for example, centrifugation, filtration, evaporation of the solvents, resuspension and dialysis methods. Thus, a powder can be obtained by removing the solvents, in particular by removing the water, for example in a spray-drying process, from the aqueous suspension of the microcapsules.

Depending on the encapsulated active ingredient, the microcapsules with a core/shell structure according to the invention are suitable as additive to detergents and cleaners, textile care compositions, cosmetics, pharmaceutical compositions, crop protectant preparations, plastics, animal feeds, foods or nutritional supplements.

The present invention further provides the use of the microcapsules with a core/shell structure which have been described above or which have been produced by the process described above as additive to detergents and cleaners, textile care compositions, cosmetics, pharmaceutical compositions, crop protectant preparations, plastics, animal feeds, foods or nutritional supplements.

The present invention further provides pulverulent or liquid preparations comprising the microcapsules with a core/shell structure described above or which have been produced by the process described above.

Besides the microcapsules with a core/shell structure, the pulverulent or liquid preparations usually comprise at least one of the customary additives and/or auxiliaries which are known to the person skilled in the art for the particular area of application, for example in the field of detergents and cleaners, textile care compositions, cosmetics, pharmaceutical compositions, crop protectant preparations, plastics, animal feeds, foods or nutritional supplements.

The present invention likewise provides the use of the pulverulent or liquid preparations described above as additive to detergents and cleaners, textile care compositions, cosmetics, pharmaceutical compositions, crop protectant preparations, plastics, animal feeds, foods or nutritional supplements.

The present invention further provides detergents and cleaners, textile care compositions, cosmetics, pharmaceutical compositions, crop protectant preparations, plastics, animal feeds, foods or nutritional supplements comprising the microcapsules with a core/shell structure according to the invention which have been described above or which have been produced by the process described above.

The invention is illustrated by the examples below, although these do not limit the invention.

### EXAMPLES

### Example 1

### Encapsulation of citronellyl nitrile (CAS Reg. No. 51566-62-2) with zinc oxide

20 g of citronellyl nitrile (BASF SE) were dissolved at room temperature (20° C.) in a mixture of 7.5 g of paraffin oil and 2.5 g of glycerol monooleate (Mazol® PGO 31 K from BASF Corporation). 4 g of polyoxyethylene(20) sorbitan monolaurate (polysorbate 20, commercially available under Tween® 20) and 4 g of alkylphenol ether sulfate (Lutensit® A-ES from BASF SE) were dissolved in 150 g of a 0.5 molar, aqueous solution of zinc chloride. The oil phase was homogenized with the water phase using an ultrasound rod (sonotrode 14 mm) for 3 minutes. The emulsion was stirred for 2 hours at room temperature in order to complete the formation of the zinc ion hydrate sheaths around the oil droplets. Free salt and excess surfactant were washed out of the emulsion by means of cross-flow filtration until a value for the conductivity of the emulsion of 0.5 mS/cm was achieved. 75 ml of a 1 molar aqueous solution of NaOH which comprised polyacrylic acid sodium salt (Sokalan® PA 15 from BASF SE) in a concentration of 5 g/l were slowly added (0.5 ml/min) to the washed emulsion in order to start the formation of zinc oxide at the surface of the oil droplets. When the addition of the NaOH- and polyacrylic acid sodium salt-containing solution was complete, the formed suspension was further stirred at 40° C. for 5 h using a magnetic stirrer in order to allow the zinc oxide shells to age.

The particle size distribution of the formed microcapsules was determined by means of light scattering in accordance with ISO 13320-1 (Microtrac S3500 Bluewave from Microtrac):

d50=0.6 μm.

The theoretical loading of the microcapsules with citronellyl nitrile is about 45% by weight, based on the mass of the microcapsules.

The actual loading of the microcapsules was determined by means of thermogravimetry (TGA). The weight loss of a sample dried at 300° C. compared to the starting sample spray-dried at 130° C. is about 30%. This means that the loading of the microcapsules is at least 30% by weight, based on the mass of the microcapsules.

### Example 2

### Encapsulation of linalyl acetate (CAS Reg. No. 115-95-7) with zinc oxide

0.6 g of polyoxyethylene(20) sorbitan monolaurate (polysorbate 20, commercially available under Tween® 20) and 0.6 g of alkylphenol ether sulfate (Lutensit® A-ES from BASF SE) were dissolved in 30 g of a 0.5 molar, aqueous solution of zinc chloride. As oil phase, 6 g of linalyl acetate (BASF SE) were homogenized together with the water phase using an ultrasound rod (sonotrode 6 mm) for 3 minutes. The emulsion was stirred for 2 hours at 40° C. in order to complete the formation of the zinc ion hydrate sheaths around the oil droplets. Free salt and excess surfactant were washed out of the emulsion by means of cross-flow filtration until a value for the conductivity of the emulsion of 0.5 mS/cm was reached. 30 ml of a 1 molar aqueous solution of NaOH which comprised polyacrylic acid sodium salt (Sokalan® PA 15 from BASF SE) in a concentration of 5 g/l were added to the washed emulsion at a volume flow rate of 0.5 ml/min in order to start the formation of zinc oxide at the surface of the oil droplets. When the addition of the NaOH- and polyacrylic acid sodium salt-containing solution was complete, the formed suspension was further stirred for 5 h at 40° C. using a magnetic stirrer in order to allow the zinc oxide shells to age.

The particle size distribution of the formed microcapsules was determined by means of light scattering in accordance with ISO 13320-1 (Microtrac S3500 Bluewave from Microtrac):

d50=0.5 μm.

### Example 3

### Encapsulation of 2-methyl-3-(4-tert-butylphenyl)propanal (Lysmeral® Extra from BASF SE) (CAS Reg. No. 80-54-6) with zinc oxide

0.6 g of polyoxyethylene(20) sorbitan monolaurate (polysorbate 20, commercially available under Tween® 20) and 0.6 g of alkylphenol ether sulfate (Lutensit® A-ES from BASF SE) were dissolved in 30 g of a 0.5 molar, aqueous solution of zinc chloride. As oil phase, 6 g of 2-methyl-3-(4-tert-butylphenyl)propanal (Lysmeral® Extra from BASF SE) were homogenized together with the water phase using an ultrasound rod (sonotrode 14 mm) for 3 minutes. The emulsion was stirred for 2 hours at 40° C. in order to complete the formation of the zinc ion hydrate sheaths around the oil droplets. Free salt and excess surfactant were washed out of the emulsion by means of cross-flow filtration until a value for the conductivity of the emulsion of 0.5 mS/cm was reached. 30 ml of a 1 molar aqueous solution of NaOH which comprised polyaspartic acid in a concentration of 5 g/l were added to the washed emulsion at a volume flow rate of 0.5 ml/min in order to start the formation of zinc oxide at the surface of the oil droplets.

When the addition of the NaOH- and polyaspartic acid-containing solution was complete, the formed suspension was further stirred for 8 h at 40° C. using a magnetic stirrer in order to allow the zinc oxide shells to age.

The particle size distribution of the formed microcapsules was determined by means of light scattering in accordance with ISO 13320-1 (Microtrac S3500 Bluewave from Microtrac):

d50=0.8 μm.

1.-15. (canceled)

16. A microcapsule with a core/shell structure which comprises at least one low molecular weight, anionic surfactant, wherein each microcapsule comprises in the inside a core which comprises at least one sparingly water-soluble or water-insoluble organic active ingredient, and, directly around the core, has a shell, wherein the shell comprises at least one metal oxide and at least one water-soluble polymer.

17. The microcapsule according to claim 16, wherein the metal oxide of the shell is zinc oxide.

18. The microcapsule according to claim 16, wherein the sparingly water-soluble or water-insoluble organic active ingredient is an aroma chemical.

19. The microcapsule according to claim 16, wherein the microcapsule further comprises at least one nonionic surfactant besides the low molecular weight, anionic surfactant.

20. The microcapsule according to claim 16, wherein the low molecular weight, anionic surfactant is a fatty alcohol sulfate or an alkylphenol ether sulfate.

21. The microcapsule according to claim 16, wherein the water-soluble polymer comprises carboxy or carboxylate groups in the side chains.

22. The microcapsule according to claim 16, wherein the microcapsule has an average particle size of less than 2 μm.

23. The microcapsule according to claim 16, wherein the core is a liquid at 20° C.

24. A process for producing microcapsules with a core/shell structure which comprise at least one low molecular weight, anionic surfactant, wherein each microcapsule comprises in the inside a core which comprises at least one sparingly water-soluble or water-insoluble organic active ingredient, and, directly around the core, has a shell, wherein the shell comprises at least one metal oxide and at least one water-soluble polymer,
comprising the steps of
i) preparing an oil-in-water emulsion by emulsifying an oil phase which comprises at least one sparingly water-soluble or water-insoluble organic active ingredient in a water phase which comprises at least one low molecular weight, anionic surfactant and at least one water-soluble salt of a metal in oxidation state +II, +III or +IV, using shear forces,
ii) washing the oil-in-water emulsion to remove salt dissolved free in the water phase and anionic surfactant until the aqueous phase has a conductivity of less than 2.0 mS/cm,
iii) producing solid metal oxide at the oil phase boundary from the water-soluble salt of the metal in oxidation state +II, +III or +IV, which is bonded in the region of the oil/water phase boundary by adding an aqueous solution which comprises a strong base and a water-soluble polymer to the emulsion washed in process step ii), and
iv) optionally, purifying and/or isolating the microcapsules with core/shell structure produced in process step iii).

25. The process according to claim 24, wherein, in process step i), the water-soluble salt used is zinc chloride, zinc nitrate or zinc acetate.

26. The process according to claim 24, wherein, in process step iii), the aqueous solution which comprises a strong base and a water-soluble polymer is added in a uniformly distributed manner over a period from 2 to 5 hours.

27. A detergent or cleaner, textile care composition, cosmetic product, pharmaceutical composition, crop protectant preparation, plastic, animal feed, food or nutritional supplement comprising twhich comprises the microcapsules with a core/shell structure according to claim 16 .

28. A pulverulent or liquid preparation comprising the microcapsules with a core/shell structure according to claim 16.

29. A pulverulent or liquid preparation comprising the microcapsules with a core/shell structure produced by the process according to claims 24.

30. A detergent or cleaner, textile care composition, cosmetic product, pharmaceutical composition, crop protectant preparation, plastic, animal feed, food or nutritional supplement comprising the pulverulent or liquid preparations according to claim 28.

31. A detergent or cleaner, textile care composition, cosmetic product, pharmaceutical composition, crop protectant preparation, plastic, animal feed, food or nutritional supplement comprising the pulverulent or liquid preparations according to claim 29.

32. A detergent or cleaner, textile care composition, cosmetic product, pharmaceutical composition, crop protectant preparation, plastic, animal feed, food or nutritional supplement comprising the microcapsules with a core/shell structure produced by the process according to claim 24.
